(19) United States
(12) Patent Application Publication
(10) Pub. No.: US 2025/0376572 A1
(43) Pub. Date: Dec. 11, 2025

(54) DYNAMIC CONJUGATE ACCEPTOR DIOL MONOMER AND POLYURETHANE DERIVED THEREFROM

(71) Applicant: University of Massachusetts, Westborough, MA (US)

(72) Inventor: James F. Reuther, Merrimack, NH (US)

(21) Appl. No.: 19/228,986

(22) Filed: Jun. 5, 2025

Related U.S. Application Data
(60) Provisional application No. 63/656,700, filed on Jun. 6, 2024.

(51) Int. Cl. C08J 11/28; C07C 225/14; C07C 225/18; C07D 319/06; C08G 71/04

(52) U.S. Cl. CPC C08J 11/28; C07C 225/14; C07C 225/18; C07D 319/06; C08G 71/04

(57) ABSTRACT

A diol-containing monomer has the structure

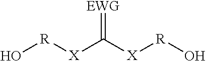

wherein X is independently at each occurrence sulfur (—S—) or nitrogen (—NH—); R is independently at each occurrence a C₁₋₁₂ alkylene group, a C₆₋₂₀ arylene group, a C₆₋₂₀ alkylarylene group, or a group of the formula —(CH₂CH₂O)_{y}CH₂CH₂—, wherein y is 1 to 4; and EWG is an electron withdrawing group. The diol-containing monomer can be particularly useful in the preparation of polyurethanes, which can be reprocessable. A method of recycling a polyurethane having repeating units derived from the diol-containing monomer is also disclosed.

### CROSS REFERENCE TO RELATED APPLICATION

This applications claims priority to U.S. Provisional Patent Application No. 63/656,700, filed on Jun. 6, 2024, the contents of which is hereby incorporated by reference in its entirety.

### FEDERAL RESEARCH STATEMENT

This invention was made with government support under award number W911QY2320006 awarded by the United States Army. The government has certain rights in the invention.

### BACKGROUND

Traditional thermoset polymeric materials, widely utilized in industries ranging from aerospace and automotive to construction and consumer goods, possess highly cross-linked molecular structures that confer excellent mechanical strength, thermal stability, and chemical resistance. However, these same properties render them inherently inflexible to reprocessing, remolding, or recycling once they have cured. Upon mechanical failure, damage, or end-of-life disposal, thermoset plastics cannot be reshaped or repurposed, unlike thermoplastic counterparts. As a result, they are typically landfilled or incinerated, contributing significantly to the accumulation of persistent plastic waste in the environment. The inability to recycle or upcycle these materials not only increases the ecological burden but also limits the sustainability of manufacturing systems that rely on them.

Accordingly, there remains a continuing need for new mechanically tough, thermoset materials that can be reprocessed, remolded, or selectively degraded to high-value byproducts for subsequent upcycling.

### SUMMARY

An aspect of the present disclosure is a diol-containing monomer of the structure

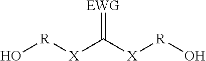

wherein X is independently at each occurrence sulfur (—S—) or nitrogen (—NH—); R is independently at each occurrence a C₁₋₁₂ alkylene group, a C₆₋₂₀ arylene group, a C₆₋₂₀ alkylarylene group, or a group of the formula —(CH₂CH₂O)_{y}CH₂CH₂—, wherein y is 1 to 4; and EWG is an electron withdrawing group.

Another aspect is a polyurethane comprising repeating units derived from the diol-containing monomer.

Another aspect is a reprocessable, crosslinked polyurethane comprising repeating units derived from the diol-containing monomer.

Another aspect is an article comprising the reprocessable, crosslinked polyurethane.

Another aspect is a method for recycling a polyurethane, the method comprising: contacting a polyurethane comprising repeating units derived from the diol-containing monomer with a decoupling agent under conditions effective to provide a polyurethane degradation product.

Another aspect is a polyurethane vitrimer composition made by a method comprising: melt-processing a mixture comprising a first polyurethane comprising repeating units derived from the diol-containing monomer, and a second polyurethane; under conditions effective to provide the polyurethane vitrimer composition.

Another aspect is a method of 3D printing, the method comprising: providing a molten composition comprising: a polyurethane comprising repeating units derived from the diol-containing monomer; and a processing aid comprising a thermoplastic polymer; extruding the molten composition to provide a fiber; forming an article from the fiber using an additive manufacturing technique.

The above described and other features are exemplified by the following figures and detailed description.

### BRIEF DESCRIPTION OF THE DRAWINGS

The following figures are exemplary embodiments.

FIG. 1 shows an exemplary chemical scheme illustrating synthesis of dynamic polyurethane materials according to an aspect of the present disclosure.

FIG. 2 shows synthesis of DCA-diol monomers with varying DCA scaffold structure to enable PU-vitrimers with different exchange kinetics and relaxation times.

FIG. 3 shows a photograph of various DCA-PU foams formed using cup-tests with 1-10 w % DCA-diol integrated.

FIG. 4 shows a photograph of degradation studies with DCA-PU foams (200 mg) in neat EDA and 1:1 EDA:THF and byproduct recovery.

FIG. 5 shows a photograph of die cut specimens from DCA-PU plaques with 1-10 w % D (left) or I (right) for tensile and tear testing.

FIG. 6A shows a plot of storage modulus, loss modulus, and complex viscosity versus temperature for various samples determined using a temperature sweep analysis.

FIG. 6B shows a plot of Tᵥ (° C.) for DCA-PU samples having varying concentrations of the DCA monomer.

FIG. 6C shows a comparison of normalized stress relaxation behavior of a control sample and DCA-PU vitrimer systems at 150° C., highlighting the absence of modulus decay in the control sample and the significant relaxation in vitrimer formulations due to dynamic covalent exchange.

FIG. 6D shows an Arrhenius plot for stress relaxation of networks derived from various DCA monomers.

FIG. 6E shows a graph depicting the calculated flow activation energies for each network.

FIG. 7 shows a photograph of a filament prepared according to an aspect of the present disclosure, and subsequent additive manufacturing of various articles using a fused filament fabrication technique.

### DETAILED DESCRIPTION

The development of reprocessable vitrimer thermosets with dynamic covalent crosslinks capable of associative bond exchange can provide thermoset materials with reprocessability. The present disclosure is directed to polyurethane (PU) vitrimer materials which can offer a route to mechanically tough, thermoset materials that can be reprocessed, remolded. or selectively degraded to high-value PU byproducts for subsequent upcycling.

The chemistry described herein utilizes dynamic conjugate addition of nucleophiles such as thiols, alcohols, and amines into specific dynamic conjugate acceptor (DCA) functional groups as the exchangeable and degradable bonds within the polymer material. Upon addition of specific chemicals (e.g., dithiothreitol, ethylene diamine, 2-mercaptoethanol, 1,2-proplyene diamine, 1,3-propylene diamine, etc.), these DCA groups are selectively cleaved and decoupled, forming a sacrificial, cyclized byproduct and releasing the macromolecular binding partners (e.g., in the form of free thiols, amines or alcohols) for subsequent chemical reactions/material development (i.e., upcycling). The polyurethane vitrimers described herein may be particularly useful in the manufacture of combat boots, providing potential routes to recyclable and upcyclable PU midsole/outsole and synthetic leather upper alternatives to increase the lifetime of boots and provide a unique chemical degradation pathway for upcycling. Additionally, it was surprisingly found that forming physical mixtures of DCA-containing polyurethanes with non-DCA-containing polyurethanes could lead to formation of a vitrimer composition after melt processing. A significant advantage is therefore provided by the present disclosure.

An aspect of the present disclosure is the synthesis and implementation of diol-containing monomers comprising DCA moieties. The monomer structures can be varied in terms of the atom connectivity to a central alkene group where dynamic exchange occurs. For example, the monomer structures can comprise a dithiol group (DCA-SS), a diamine group (DCA-NN), or an amine-thiol group (DCA-SN). The monomer structures can also be varied in terms of the spacer or linking group between the DCA unit and the alcohol functionality. Exemplary linking groups can include C₁₋₁₂ alkyl groups, alkyloxy groups, aryl groups, alkylaryl groups (e.g., benzylic groups), and the like. Additionally, the monomers can be varied in their electron withdrawing moieties to include Meldrum's acid (DCA-1), Dimedone (DCA-2), Indanodione (DCA-3), cyclic dione (of varying ring size; DCA-4-6 for z=1-3, respectively), dicyano (DCA-7), acyclic diester (DCA-8-9) and acyclic dione (DCA-10) functionalities shown below.

For example, the diol-containing monomer can be of the formula

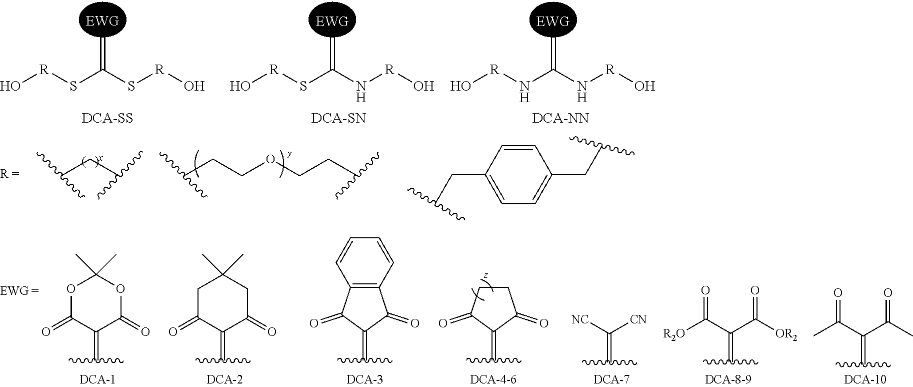

wherein x is 4 to 12, y is 1 to 4, z is 1 to 3 and R₂ is either a methyl or ethyl group. The curved lines are understood to represent the points of attachment of the R and the EWG group to the rest of the diol-containing monomer.

Thus an aspect of the present disclosure is a diol-containing monomer of the structure

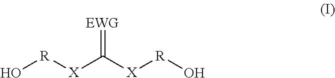

wherein X is independently at each occurrence sulfur (—S—) or nitrogen (—NH—); R is independently at each occurrence a C₁₋₁₂ alkylene group, a C₆₋₂₀ arylene group, a C₆₋₂₀ alkylarylene group, or a group of the formula —(CH₂CH₂O)_{y}CH₂CH₂—, wherein y is 1 to 4; and EWG is an electron withdrawing group. The electron withdrawing group can be selected from

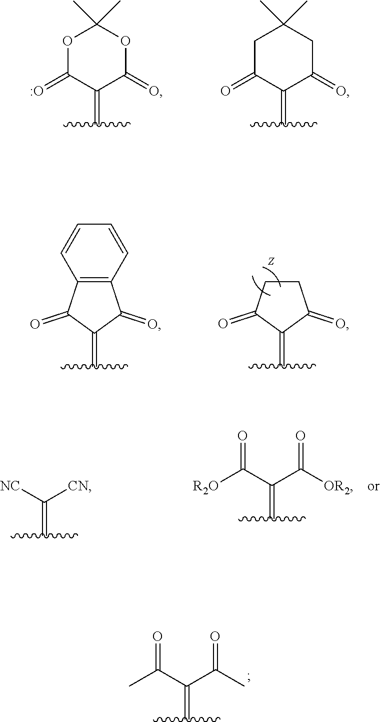

wherein z is 1 to 3; and R₂ is independently at each occurrence methyl or ethyl; wherein the curved lines represent points of attachment to the diol-containing monomer.

In some aspects, R in structure (I) can be independently at each occurrence

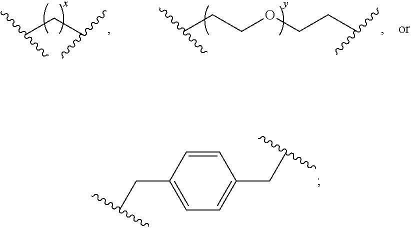

wherein x is 4 to 12; and y is 1 to 4; wherein the curved lines represent points of attachment to the diol-containing monomer.

In a specific aspect, the diol-containing monomer can have the structure

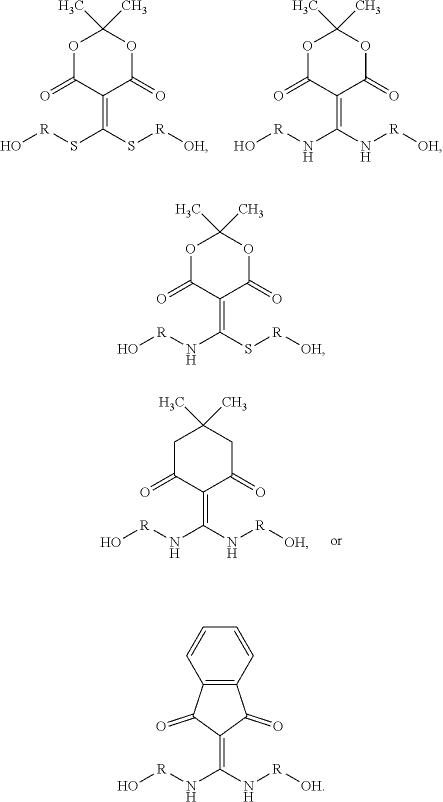

In each of the foregoing structures, R can independently at each occurrence be a C₁₋₁₂ alkylene group, a C₆₋₂₀ arylene group, a C₆₋₂₀ alkylarylene group, or a group of the formula —(CH₂CH₂O)_{y}CH₂CH₂—, wherein y is 1 to 4. In some aspects, each occurrence of R can independently be a C₁₋₁₂ alkylene group, or a C₁₋₆ alkylene group, or a C₃₋₆ alkylene group. In some aspects, each occurrence of R in a given diol-containing monomer can be the same. In some aspects, each occurrence of R in a given diol-containing monomer can be different.

Another aspect of the present disclosure is the synthesis and implementation of polyurethane formulations prepared by (co)polymerization of the above-described diol-containing monomer. Accordingly, a polyurethane comprising repeating units derived from the diol-containing monomer of the present disclosure represents another aspect. In some aspects, the polyurethane can be a homopolymer (i.e., consisting of repeating units derived from the diol-containing monomer and a diisocyanate) or a copolymer (i.e., having repeating units derived from the diol-containing monomer and a diisocyanate, and one or more additional diol-containing monomers).

In particular, the diol-containing monomer of the present disclosure can be polymerized with a diisocyanate (or an oligomer thereof), and optionally a second diol-containing monomer to provide the polyurethane.

Diisocyanates for the manufacture of polyurethanes are generally known, and any suitable diisocyanate may be used herein provided that the resulting polyurethane does not exhibit an undesirable property due to inclusion of a particular diisocyanate. For example, the diisocyanate can be aromatic, aliphatic, or cycloaliphatic. Representative diisocyanates can include, but are not limited to toluene diisocyanate (TDI), including the 2,4- and 2,6-isomers; methylene diphenyl diisocyanate (MDI), including 4,4′-MDI, 2,4′-MDI, and polymeric MDI (pMDI); naphthalene diisocyanate (NDI); and p-phenylene diisocyanate (PPDI). Representative aliphatic diisocyanates include hexamethylene diisocyanate (HDI), isophorone diisocyanate (IPDI), tetramethylene diisocyanate (TMDI), and trimethylhexamethylene diisocyanate. Suitable cycloaliphatic diisocyanates may include dicyclohexylmethane diisocyanate and cyclohexylene diisocyanate. These diisocyanates may be employed individually or in various combinations, depending on the desired physical and chemical properties of the resulting polyurethane.

In a specific aspect, the diisocyanate can comprise toluene diisocyanate, methylene diphenyl diisocyanate, or a combination thereof.

In some aspects, a second diol-containing monomer can be provided. Suitable diols for use in the formation of polyurethanes include, but are not limited to, ethylene glycol, diethylene glycol, 1,4-butanediol, 1,3-propanediol, 1,6-hexanediol, neopentyl glycol, and cyclohexanedimethanol. Dihydroxyl-terminated polymers or oligomers can also be used. Suitable hydroxyl-functionalized polymers and oligomers for use in the formation of polyurethanes include, but are not limited to, polyether polyols (such as polyethylene glycol, polypropylene glycol, and polytetramethylene ether glycol), polyester polyols (such as those derived from adipic acid, phthalic anhydride, and sebacic acid), polycarbonate polyols, polycaprolactone diols, castor oil, and hydroxyl-terminated polybutadiene. These polymeric diols may be selected based on molecular weight, hydroxyl functionality, and the desired mechanical and thermal properties of the resulting polyurethane The foregoing diols may be employed individually or in combination, depending on the desired physical and mechanical properties of the resulting polyurethane. The second diol-containing monomer is not limited to these examples, and can be selected based on desired properties of the polyurethane product.

In a specific aspect, the second diol-containing monomer can include, for example, poly(tetramethylene glycol), hydroxy-terminated polybutadiene, and the like, or a combination thereof.

In some aspects, a crosslinker can be present, and the resulting polyurethane can be a crosslinked polyurethane. The crosslinker is not particularly limited and may be, for example, a polyol (e.g., having more than two alcohol groups, for example, a triol, a tetraol, etc.), a polyamine (e.g., having more than two amine groups, for example, a triamine, tetraamine, etc.), a polythiol (e.g., having more than two thiol groups, for example, a trithiol, a tetrathiol, etc.), and the like, or a combination thereof.

The DCA-diol-containing monomer, the diisocyanate, and (when present) the second diol-containing monomer and crosslinker can be present in varying molar ratios and can be selected depending on the desired mechanical properties, reprocessability, and degradation products. For example, a molar ratio of the DCA-diol-containing monomer to the diisocyanate, the second diol-containing monomer, and the crosslinker can be 1:99 to 99:1 by weight, or 5:95 to 95:5, or 10:90 to 90:10, or 15:85 to 85:15, or 20:80 to 80:20, or 25:75 to 75:25, or 30:70 to 70:30, or 40:60 to 60:40, or 45:55 to 55:45. The working examples below illustrate an exemplary polymerization of a DCA-diol-containing monomer, a second diol-containing monomer, and a diisocyanate/diisocyanate oligomer (crosslinker), followed by crosslinking.

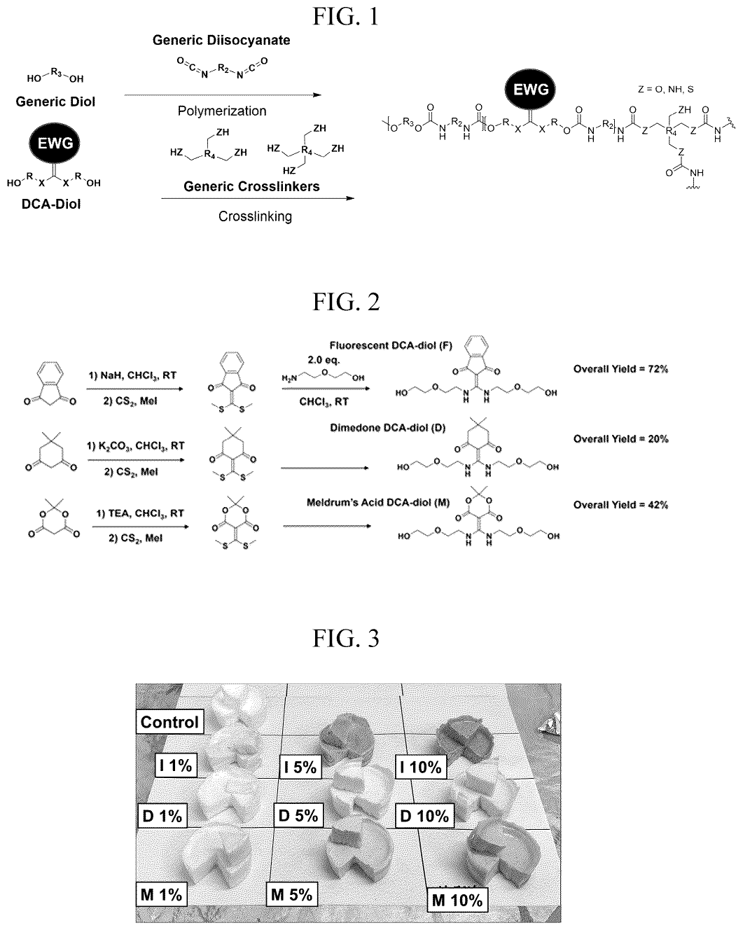

The typical process for synthesis of dynamic PU materials would include dissolving DCA-diol monomers in resin formulations and mixing with isocyanate formulations (also can be commercially available) to attain unique properties. An illustrative chemical synthesis is shown in FIG. 1.

In some aspects, the polyurethane can be foamed. Foamed polyurethanes are cellular polymeric materials formed by the reaction of the monomeric materials described herein in the presence of a blowing agent. The blowing agent, which may be chemical (e.g., water reacting with isocyanate to produce carbon dioxide) or physical (e.g., volatile hydrocarbons, hydrofluorocarbons, carbon dioxide, etc.), creates a gas phase during polymerization, resulting in a foam structure. The resulting material may be either flexible or rigid depending on the formulation, including the choice of diol, isocyanate, crosslinker, any additives that may be present, and cell structure, size, and density.

Advantageously, the dynamic structure of the diol-containing monomers described herein allow for the polyurethanes comprising repeat unit derived from these monomers to be crosslinked, but also reprocessable.

The polyurethanes described herein can be used for a variety of applications. For example, articles comprising a polyurethane can include flexible and rigid foams for furniture, mattresses, and thermal insulation panels; elastomeric components such as wheels, tires, seals, gaskets, and vibration dampeners; coatings, adhesives, sealants, and binders for use in construction, automotive, and industrial applications; textile laminates, synthetic leather, and footwear components; medical devices such as wound dressings and catheters; automotive interior components including seats, headrests, and dashboards; electronic encapsulants and protective casings; and packaging materials including protective cushioning and spray foams. The selection of polyurethane formulation may vary depending on the mechanical, thermal, chemical, and aesthetic requirements of the intended article. In a specific aspect, the polyurethane of the present disclosure may be particularly well suited for use as a footwear component, such as a shoe sole. In some aspects, the polyurethane can be used as protective padding, for example helmet padding, or can be used as ear protection, for example earplugs.

The polyurethanes of the present disclosure are also particularly well suited for improved recyclability. Accordingly, another aspect of the present disclosure is a method for recycling a polyurethane. The method comprises contacting a polyurethane of the present disclosure (i.e., comprising repeating units derived from the particular diol-containing monomers described herein) with a decoupling agent.

As used herein, a “decoupling agent” refers to a chemical species that facilitates the cleavage of covalent bonds within a polymer backbone or cross-linked network under controlled conditions, thereby enabling depolymerization, degradation, or chemical recycling of the polymer. The decoupling agent may act by selectively breaking dynamic covalent bonds of the diol-containing monomers of the present disclosure that were intentionally incorporated into the polymer structure during synthesis. In some aspects, the decoupling agent may be a nucleophile, electrophile, acid, base, reductant, oxidant, or catalytic species capable of initiating or accelerating bond scission reactions.

In some aspects, the decoupling agent can be any suitable compound having at least one nucleophile, such as a thiol group, a hydroxyl group, an amine group, or a combination thereof. For example, in an aspect, the decoupling agent can comprise water; alcohols such as methanol, ethanol, 1-butanol, benzyl alcohol, ethylene glycol, 1,4-butanediol, and glycerol; thiols such as mercaptoethanol, thioglycolic acid, 1,2-ethanedithiol, cysteamine, and glutathione; and amines such as ethylenediamine, hexamethylenediamine, monoethanolamine, diethanolamine, piperazine, and triethylenetetramine.

In some aspects, the polyurethane degradation product can be used to form a new polymer network. For example, in some aspects, the polyurethane degradation product comprises amine functional groups at the chain end, which can be reacted with a diisocyanate to form a new polyurethane product, also referred to herein as an upcycled polyurethane.

In some aspects, the polyurethane degradation product can be further functionalized. For example, reaction of the amine-diterminated polyurethane degradation product with a variety of electrophilic reagents to introduce terminal functional groups. Suitable compounds include, but are not limited to, acid chlorides (e.g., methacryloyl chloride, acryloyl chloride, benzoyl chloride), anhydrides (e.g., maleic anhydride, succinic anhydride, phthalic anhydride), isocyanates (e.g., isophorone diisocyanate, toluene diisocyanate, hexamethylene diisocyanate) in combination with hydroxy-functional compounds such as hydroxyethyl methacrylate, or epoxy-functional compounds (e.g., glycidyl methacrylate). These reactions result in the introduction of functional end groups including (meth)acrylate, carboxyl, urethane, or epoxy moieties, enabling further crosslinking or copolymerization reactions for various applications. For example, including a functional group having ethylenic unsaturation (e.g., a (meth)acryl group) will enable use of the polyurethane degradation product in other polymerization processes, such as photocuring (e.g., using free radical initiators, or photoacid or photobase generators), or additive manufacturing using digital light processing (DLP) techniques. In some aspects, the polyurethane degradation product can be reacted with a suitable compound to regenerate the original polyurethane network via dynamic conjugate addition. An exemplary compound capable of reacting with the polyurethane degradation products to regenerate a polyurethane network is of the structure

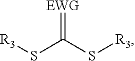

wherein EWG is as already defined herein, and R₃ is independently at each occurrence a C₁₋₁₂ alkyl group, a C₆₋₂₀ aryl group, or a C₆₋₂₀ alkylaryl group. In an aspect, R₃ can be a C₁₋₆ alkyl group, for example methyl or ethyl. In a specific aspect, each occurrence of R₃ can be methyl.

In another advantageous feature, the polyurethane comprising repeating units from the diol-containing monomer according to the present disclosure can be mixed with a conventional, non-processable polyurethane material and melt mixed to provide a reprocessable composition. Accordingly, another aspect of the present disclosure is a polyurethane vitrimer composition. The polyurethane vitrimer composition is provided by melt-processing a mixture comprising a first polyurethane and a second polyurethane. The first polyurethane comprises repeating units derived from the diol-containing monomer described herein.

The second polyurethane can generally be any polyurethane provided that it does not comprise repeating units derived from the diol-containing monomer of the present disclosure. For example, the second polyurethane can include thermoplastic polyurethanes (TPUs), typically prepared from aromatic or aliphatic diisocyanates combined with polyether or polyester polyols, used in flexible films, hoses, and footwear; cast polyurethanes commonly formed from aromatic diisocyanates, such as toluene diisocyanate (TDI) or methylene diphenyl diisocyanate (MDI), reacted with poly(1,4-butylene adipate) or poly(tetramethylene ether) glycol, widely used in industrial wheels, rollers, and seals; flexible polyurethane foams produced from polymeric MDI or TDI with polyether polyols, and are widely utilized in furniture, automotive seating, and bedding; rigid polyurethane foams, based on polymeric MDI and low-molecular-weight polyether polyols, applied in construction, insulation, and packaging; and waterborne polyurethane dispersions derived from aliphatic diisocyanates, such as hexamethylene diisocyanate (HDI) or isophorone diisocyanate (IPDI), combined with hydrophilic diols or polyols, commonly used in environmentally friendly coatings and adhesives. The second polyurethane may be a virgin material, waste material, or recycled material.

Melt-processing the mixture can generally be by any melt-processing technique such as extrusion (including single-screw and twin-screw extrusion), injection molding, blow molding, compression molding, calendering, and melt spinning. These methods enable the fabrication of articles from the mixture with various shapes and dimensions, including films, sheets, fibers, tubes, and complex molded parts, by subjecting the mixture to elevated temperatures and shear forces under controlled conditions.

The conditions effective to provide the polyurethane vitrimer composition can include, for example, melt-mixing the mixture at a temperature of 150 to 200° C., for example 160 to 190° C., or 170 to 190° C., or 175 to 185° C. In some aspects, the composition can be immediately extruded (or otherwise processed) immediately following melt-mixing. In some aspects, a dwell time may be incorporated. The dwell time (or residence time) refers to the duration of time that the mixture spends within the heated barrel of an extruder (i.e., prior to being extruded). In some aspects a dwell time of 1 to 10 minutes, or 1 to 7 minutes, or 2 to 7 minutes, or 2 to 5 minutes, or 3 to 7 minutes, or 3 to 5 minutes. It will be appreciated that other mixing conditions not specifically disclosed herein may also provide the polyurethane vitrimer composition. It will also be appreciated that parameters such as temperatures and dwell time may vary based on specific equipment configuration.

The mixture from which the polyurethane vitrimer is prepared can comprise 1 to 99 weight percent of the first polyurethane, based on the total weight of the mixture. Within this range, the mixture can comprise at least 10 weight percent, or at least 15 weight percent, or at least 20 weight percent, or at least 30 weight percent, or at least 40 weight percent, or at least 50 weight percent, or at least 60 weight percent, or at least 70 weight percent, or at least 80 weight percent, or at least 90 weight percent of the first polyurethane. Also within this range, the mixture can comprise at most 90 weight percent, or at most 85 weight percent, or at most 80 weight percent, or at most 70 weight percent, or at most 60 weight percent, or at most 50 weight percent, or at most 40 weight percent, or at most 30 weight percent, or at most 20 weight percent, or at most 10 weight percent of the first polyurethane.

The mixture from which the polyurethane vitrimer is prepared can comprise 1 to 99 weight percent of the second polyurethane, based on the total weight of the mixture. Within this range, the mixture can comprise at least 10 weight percent, or at least 15 weight percent, or at least 20 weight percent, or at least 30 weight percent, or at least 40 weight percent, or at least 50 weight percent, or at least 60 weight percent, or at least 70 weight percent, or at least 80 weight percent, or at least 90 weight percent of the second polyurethane. Also within this range, the mixture can comprise at most 90 weight percent, or at most 85 weight percent, or at most 80 weight percent, or at most 70 weight percent, or at most 60 weight percent, or at most 50 weight percent, or at most 40 weight percent, or at most 30 weight percent, or at most 20 weight percent, or at most 10 weight percent of the second polyurethane.

The polyurethane vitrimer according to the present disclosure exhibits improved mechanical properties compared to second polyurethane in the absence of any of the first polyurethane (i.e., when no DCA-containing repeating units are present). For example, the polyurethane vitrimer of the present disclosure exhibits at least one of tensile stress at maximum force, tensile strain at break, tensile stress at break, displacement at break, and Young's modulus that is improved relative to the corresponding property for the second polyurethane alone.

Another aspect of the present disclosure is a method of 3D printing using the polyurethane comprising repeating units derived from the diol-containing monomer described herein. 3D printing, also known as additive manufacturing, provides the ability to create advanced three-dimensional structures. There are many types of additive manufacturing techniques. One type of additive manufacturing is known as fused filament fabrication, in which a polymeric filament is used to generate a print line through melt extrusion.

The method according to the present disclosure comprises providing a molten composition. The molten composition comprises a polyurethane comprising repeating units derived from the diol-containing monomer of the present disclosure, and a processing aid comprising a thermoplastic polymer.

The processing aid comprising the thermoplastic polymer can be selected such that the molten composition is in the melt state during the extrusion, but is solid at ambient temperature (for example 20-25° C.) and such that the solidified composition possesses a described combination of mechanical properties. Thermoplastic polymer processing aids may also be incorporated to enhance the melt flow, reduce processing temperatures, minimize die buildup, and improve surface finish. Suitable thermoplastic polymer processing aids are characterized by their ability to soften upon heating and solidify upon cooling, thereby enabling precise layer-by-layer deposition during an additive manufacturing process.

The polymer processing aid is not particularly limited and can be, for example, fluoropolymer-based processing aids, such as polytetrafluoroethylene (PTFE) and fluoroelastomers; acrylic-based processing aids, such as polymethyl methacrylate (PMMA) and polyacrylate copolymers; silicone-based processing aids, including polydimethylsiloxane (PDMS) and organosiloxane copolymers; and other thermoplastic polymers such as acrylonitrile-butadiene-styrene copolymers (ABS), polylactic acid (PLA), polyethylene terephthalate glycol (PETG), polycarbonate (PC), nylon (polyamide), thermoplastic polyurethane (TPU), high-impact polystyrene (HIPS), polypropylene (PP), and polyether ether ketone (PEEK), and the like, or combinations thereof.

The molten compositions of the present disclosure are solid at ambient temperature but fluid at an operating temperature of an additive manufacturing process. Different additive manufacturing process utilize different operating temperatures, for example falling within the range of 50 to 300° C. In some aspects, the compositions of the present disclosure become fluid at a temperature greater than 50° C., or 75° C., or 100° C., or 125° C., or 150° C., or 175° C., or 200° C., or 225° C., or 250° C., or 275° C., or 300°, including ranges thereof. For example, temperatures of 50 to 250, or 50 to 200, or 50 to 150° C. are mentioned.

The polyurethane and the processing aid can be present in the mixture in a weight ratio of, for example, 10:90 to 90:10, 20:80 to 80:20, 30:70 to 70:30, 40:60 to 60:40, 45:55 to 55:45, 48:52 to 52:48, or 50:50.

The method further comprises extruding the molten composition to provide a fiber, and forming an article (i.e., a three dimensional article) from the fiber using an additive manufacturing technique. The additive manufacturing technique can generally be any extrusion based additive manufacturing technique such as fused filament fabrication (FFF). In FFF, the fiber (or filament) is extruded through a heated nozzle and deposited layer-by-layer onto a build platform to create the three-dimensional object. The filament is continuously fed into the heated extrusion head, melted, and precisely deposited according to digital model data, allowing for the controlled fabrication of complex geometries. Each successive layer bonds to the previous layer upon cooling, resulting in a solidified final part.

Accordingly, the present disclosure addresses the technical limitations associated with known thermoset materials by introducing new, mechanically robust polymer systems that incorporate dynamic covalent chemistries, enabling their reusability and upcyclability without sacrificing performance. Furthermore, the presently disclosed polymer systems based on the dynamic covalent bonds can be used to impart reprocessability to previous non-reprocessable materials, as well in filament-based additive manufacturing processes. A significant improvement is therefore provided by the present disclosure.

### EXAMPLES

An exemplary synthesis of 3 DCA-diol monomers is shown in FIG. 2. Specifically, indanedione-derived DCA-diol (I), Dimedone-derived DCA-diol (D) and Meldrum's Acid-derived DCA-diol (M) were synthesized at scales ranging from 20-60 g of starting material with 2 step syntheses without the need for column chromatography.

Preliminary synthetic investigations were carried out integrating DCA-diol monomers using cup-tests. This approach allows for rapid prototyping to determine specific synthetic parameters associated with reaction times (i.e., cream time, gel time, top-of-cup time, etc.). Initial experiments were conducted with the foam resin listed in Table 1 incorporating 1-10 w % of each DCA-diol monomer.

TABLE 1

| | Wt % | |
|---|---|---|
| Chemical Component | incorporation | Role |
| 1,4-butanediol | 10-15% | Diol monomer |
| 2,2′-diethanolamine | 0.2-1.0% | Diamine monomer |
| DABCO | 0.3-1.0% | Amine catalyst |
| 1-methylimidazole | 0.2-1.0% | Amine catalyst |
| Tetramethy1-2,2′- | 0.2-1.0% | Blowing catalyst/ |
| oxybis(ethylamine) | | surfactant |
| MDI | 25-50% | Isocyanate monomer |
| Methylenediphenyl | 0.3-7.0% | Isocyanate monomer |
| diisocyanate | | |
| MDI Oligomers | 0.3-7.0% | Isocyanate crosslinker |
| Water | 0.57% | Blowing Agent |
| DCA-diol monomer | 1-10% | Diol monomer |

Overall, the reaction kinetics remained largely constant, and the monomers were fully soluble in the resin mixture. All reactions produced foams with cream times=30 sec (i.e., time before foaming occurs) and gel times=60 sec (i.e., time prior to solidification via curing). In this trial, a series of 9 prototype foams and 1 control sample (absent of DCA-diol additions) were fabricated, as shown in FIG. 3.

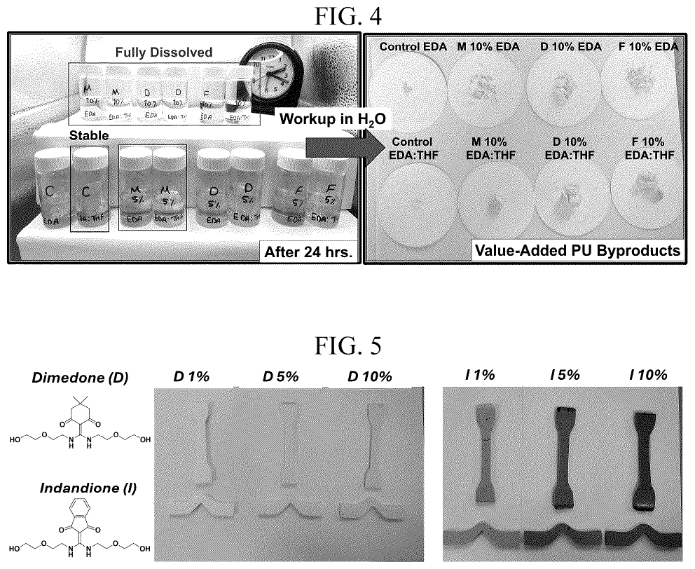

Initial studies with prototype DCA-PU foams included ethylene diamine (EDA) induced degradation of foams compared to controls. This thermoset degradation reaction is designed to occur selectively only at the DCA functional groups via EDA-DCA cyclization reactions. This results in soluble, value-added PU-byproducts terminated with amines which can be utilized in various upcycled applications. Two different conditions for degradation were used: 1) neat EDA and 2) 1:1 EDA:THF. Each sample was left standing (no agitation) at room temperature for a period of 48 hours using 200 mg of foam, as shown in FIG. 4. To monitor this, time-stop studies were conducted and compiled in video format using a mounted smart phone. DCA-M10%-PU materials dissolved (i.e., chemically degraded DCA bonds) the quickest with full dissolution in t=2 h in neat EDA and t=11 h in 1:1 EDA:THF. All DCA-PU foams with 10 w % DCA-diol were fully dissolved in EDA or EDA:THF within 24 h. Importantly, control PU foams with no DCA-diol showed little to no change over the course of 48 h in 1:1 EDA:THF, although some dissolution and degradation was observed in neat EDA. At the point of complete dissolution, each sample was removed from filming and precipitated in H₂O to isolate PU-byproducts for upcycling experiments. In each case, quantitative recovery of material was obtained with control samples in EDA:THF resulting in only ˜5 mg of precipitate (˜2.5% mass recovery) supporting full stability. Additional studies of degradation of compression molded plaques and lower concentrations of EDA (i.e., 10-40%) showed enhanced selectivity and surprisingly faster degradation rates for DCA-PU materials.

DCA-PU plaques (12″×6″×0.5″) were prepared for subsequent mechanical testing. This was performed incorporating 1-10 w % of each DCA-diol monomer (FIG. 5) into 9 testable prototypes. In all cases, the DCA-diol monomer showed good compatibility creating homogenous and mechanically robust PU-plaques. These were die cut to 3×dumbbell and 3× die C specimens according to ASTM D624-00 and ASTM D3574-17 for tensile and tear tests, respectively. The remaining portions of the plaque were also salvaged to be used for DMA, rheological, reprocessing and degradation studies.

The incorporation of DCA into PU-vitrimers generally led to a reduction in Young's modulus, indicating a decrease in stiffness. However, DCA-PU-D5% and D10% exhibited a slight increase (E=4.52 and 4.62 MPa, respectively), suggesting that DCA-D diol enhances rigidity. While increased stiffness may be beneficial for structural applications, it may be less desirable for footwear applications, where excessive rigidity could impact comfort and flexibility. Tensile stress at break, a measure of the maximum stress a material can withstand before failure, generally decreased with increasing DCA-diol content. However, DCA-M diol incorporation showed an exception, with tensile stress at break increasing with higher concentrations, suggesting that DCA-M enhances the mechanical strength of PU foams.

Tensile strain at break, which quantifies material elongation before failure, increased with DCA concentration. Notably, M10% exhibited the highest elongation at break (247.59%), indicating superior flexibility and resilience. These properties make M10% a strong candidate for applications requiring both high elongation and durability, such as footwear soles.

A comparison of tensile properties across all samples averaged from triplicate experiments is shown in Table 2.

TABLE 2

| | | Standard | Tensile | Tensile | |
|---|---|---|---|---|---|
| | Young's | Deviation of | strain | stress at | Displacement |
| | Modulus | Young's | at Break | Break | at Break |
| | (MPa) | Modulus | (%) | (MPa) | (mm) |
| Control | 4.34 | 0.11 | 220.00 | 2.92 | 198 |
| M 1% | 4.25 | 0.06 | 235.06 | 3.11 | 211.55 |
| M 5% | 3.54 | 0.05 | 229.87 | 2.83 | 206.89 |
| M 10% | 3.16 | 0.02 | 247.59 | 2.63 | 222.83 |
| I 1% | 3.36 | 0.05 | 220.86 | 2.67 | 198.78 |
| I 5% | 2.95 | 0.13 | 124.26 | 1.03 | 111.83 |
| I 10% | 3.07 | 0.08 | 108.1 | 1.05 | 97.28 |
| D 1% | 4.15 | 0.08 | 216.41 | 2.74 | 194.78 |
| D 5% | 4.52 | 0.02 | 227.59 | 2.58 | 204.83 |
| D 10% | 4.62 | 0.08 | 124.93 | 1.39 | 112.44 |

Tear testing of die-C cut samples was conducted in accordance with ASTM D624 to evaluate the tear resistance of DCA-PU vitrimers. Three key parameters were analyzed: displacement at tear, force at tear, and tear strength, providing insight into the influence of DCA-diol concentration and structure on material resilience. Tear test results are shown in Table 3.

TABLE 3

| | Average | | Average |
|---|---|---|---|
| | Displacement | Average | Tear |
| | at Tear | Force at | Strength |
| | (mm) | Tear (N) | (N/m) |
| Control | 32.08 | 84.72 | 7451 |
| M 1% | 33.41 | 91.26 | 7970 |
| M 5% | 41.41 | 98.44 | 8685 |
| M 10% | 45.58 | 96.70 | 8584 |
| I 1% | 38.00 | 90.90 | 7932 |
| I 5% | 37.91 | 57.03 | 5207 |
| I 10% | 45.83 | 67.21 | 5902 |
| D 1% | 42.66 | 94.98 | 8450 |
| D 5% | 49.41 | 102.59 | 8960 |
| D 10% | 26.50 | 61.00 | 5383 |

Force at tear, which measures the maximum force required to initiate tearing, varied across formulations. DCA-PU-D-5% exhibited the highest tear force (102.59 N), indicating superior tear resistance, whereas DCA-PU-J-5% and DCA-PU-D-10% showed the lowest values (57.03 N and 61.00 N, respectively). These results suggest that moderate vitrimer concentrations (5%) may enhance tear resistance, while higher concentrations (10%) may reduce force tolerance due to increased chain mobility. Notably, DCA-M diol incorporation consistently improved tear force across all samples demonstrating the superior performance of DCA-M for maximizing mechanical properties.

Tear strength was calculated using the equation: Tear Strength=Force at Tear/Thickness. With an average sample thickness of 6.67 mm, the control exhibited a tear strength of 7451 N/m. Most DCA-PU formulations containing 1-5 mol % DCA-diol demonstrated higher tear strengths, with the exception of DCA-PU-I-5%. However, at 10% DCA-diol content, tear strength significantly decreased for DCA-PU-I-10% and DCA-PU-D-10% (˜5900 N/m and 5400 N/m, respectively). DCA-M diol remained an exception, maintaining enhanced tear strength at higher concentrations.

Overall, DCA-M monomers significantly improved mechanical properties while potentially enabling vitrimer characteristics that promote end-of-life reprocessability and recyclability. Ongoing rheological studies are assessing the stress relaxation behavior of these materials to confirm vitrimer-like properties, with preliminary results supporting their dynamic covalent network behavior.

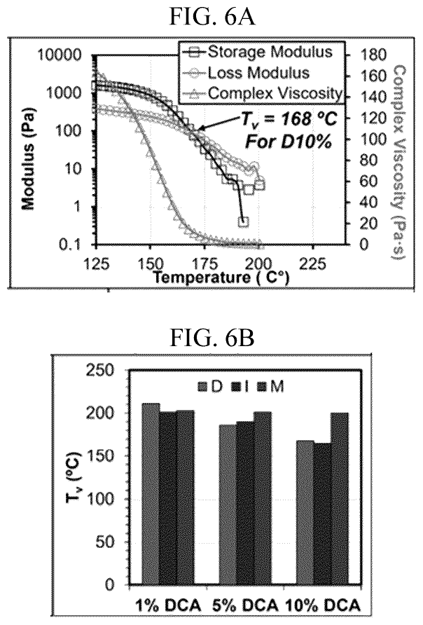

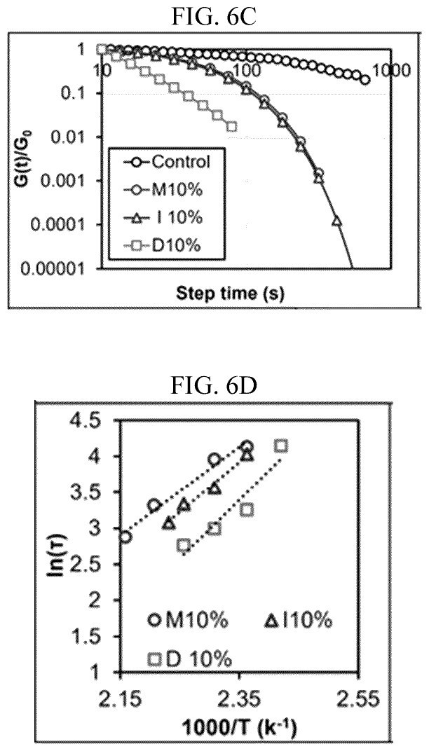

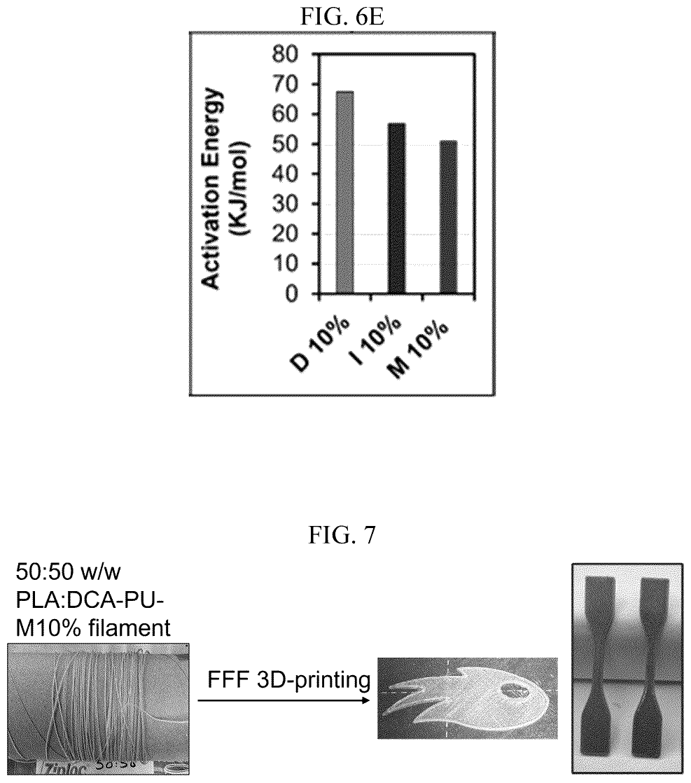

DCA-PU vitrimer materials were extensively characterized using parallel-plate rheology to identify important, vitrimer-specific properties. Temperature-sweep experiments were used to identify topology-freezing temperatures (Tᵥ) for all DCA-PU samples (example given in FIG. 6A). This temperature denotes the point dynamic exchange of DCA bonds becomes sufficiently rapid to permit flowability/reprocessability and can be likened to a melting-transition for thermoplastics. For this, the crossover of storage (G′) and loss (G″) modulus was selected as the Tᵥ for each sample (FIG. 6A) and plotted in the bar graph in FIG. 6B, showing a dependency on monomer composition and % incorporation. This data is compiled in Table 4.

TABLE 4

| | 1% DCA | 5% DCA | 10% DCA |
|---|---|---|---|
| DCA | Tᵥ (° C.) | Tᵥ (° C.) | Tᵥ (° C.) |
| D | 211 | 186 | 168 |
| I | 201 | 190 | 165 |
| M | 203 | 201 | 200 |

Performing stress-relaxation analysis at various temperatures demonstrates the relaxation vitrimers can experience under shear unlike traditional thermosets. This data can be fit to the Maxwell model and Arrhenius analysis can be performed to calculate activation energies associated with the dynamic exchange. A summary plot of stress relaxation is given in FIG. 6C comparing DCA-PU-M10%, DCA-PU-D10%, and DCA-PU-I10% along with the control PU without DCA. FIG. 6D shows Arrhenius plots for stress relaxation of various networks. All DCA-PU vitrimers demonstrated stress relaxation and activation energies (shown in FIG. 6E) were calculated showing the lowest values for DCA-M and highest for DCA-D. This data is tabulated in Table 5.

TABLE 5

| | Activation |
|---|---|
| | Energy |
| PU-Vitrimer | (kJ/mol) |
| DCA-PU-D10% | 67.5 |
| DCA-PU-I10% | 56.8 |
| DCA-PU-M10% | 51.0 |

Polyurethanes (PUs) are widely used thermoset polymers, but their chemical cross-linking prevents melt-reprocessing, limiting recyclability. Existing recycling methods, such as mechanical rebonding and catalyzed glycolysis, often result in downcycling rather than true circularity. Previous approaches have explored covalent adaptable networks (CANs) to enable reprocessability, which are not functioning as drop-in solutions. Furthermore, dynamic linkages enabling reprocessability are not typically found in commercial thermosets, necessitating the development of new materials in an industry that has yet to fully prioritize circularity and sustainability. Other approaches include catalyst-assisted carbamate exchange or solvent-free decrosslinking with small-molecule carbamates. However, these methods either require solvents and catalysts, reducing industrial viability, or fundamentally alter PU structures, making them less compatible with existing commercial products.

In contrast, the present approach introduces a drop-in monomer strategy for synthesis of recyclable DCA-PU, which is entirely solvent- and catalyst-free. By incorporating DCA-diol monomers into commercial PU formulations, foams were synthesized that exhibit dynamic reprocessability. Rheological studies and stress relaxation tests confirmed the vitrimer behavior of these materials. After cryo-milling or cutting the vitrimer foams into small fragments, they were extruded into filaments by micro-compounder (Xplore Micro-compounder) and twin-screw extrusion. These filaments demonstrated excellent mechanical integrity and could be pelletized (Bay Plastics Machinery BT25×) and re-extruded multiple times without significant degradation.

It was also surprisingly found that conventional, non-degradable, non-processable polyurethanes could be physically mixed with the recyclable DCA-PU according to the present disclosure to induce vitrimerization, rendering the previously non-processable polyurethanes processable.

To test this, polyurethane waste material was combined with DCA-PU-M5% and M10% in varying weight ratios. The physical mixtures were extruded using a twin screw extruder. All samples including the DCA-PU showed impressive reprocessability and mechanical properties. Exemplary results are shown in Table 6.

TABLE 6

| | | Tensile | | Tensile | | |
|---|---|---|---|---|---|---|
| | | Stress @ | Tensile | Stress at | Displacement | Young's |
| | Meldrum's | Max. Force | Strain at | Break | at Break | Modulus |
| Ratio | Acid | (MPa) | Break (%) | (MPa) | (mm) | (MPa) |
| 100:0 | (control | 1.74 | 72.35 | 1.67 | 38.19 | 9.24 |
| | PU only) | | | | | |
| 50:50 | M5% | 4.68 | 261.26 | 4.46 | 132.91 | 11.36 |
| 90:10 | M5% | 5.25 | 340.17 | 5.06 | 172.96 | 12.68 |
| 50:50 | M10% | 5.36 | 323.19 | 5.14 | 164.14 | 11.85 |
| 90:10 | M10% | 6.42 | 421.07 | 6.11 | 213.54 | 13.21 |

To test the limits of the approach, physical mixing of DCA-PU vitrimers with control PU materials of the same formulation but absent of DCA chemistry were also tested. These control materials were previously shown to be non-processable via twin-screw extrusion. Physical blending of ground DCA-PU vitrimers with control PU enabled extrusion of filaments even using DCA-PU-M1% at ratios of vitrimer:control of 10:90 and 1:99. Post-processing, tensile properties of the filaments were tested using the same methodology previously described still showing substantial tensile strength. These tensile properties are detailed below in Table 7.

TABLE 7

| | | Tensile | | Tensile | | |
|---|---|---|---|---|---|---|
| | | Stress at | Tensile Strain | stress at | Displacement | |
| | | Maximum | (Displacement) | break | at Break | Young's |
| | | Force | at Break | (standard) | (Standard) | Modulus |
| Ratio | DCA-PU | [MPa] | (standard) [%] | [MPa] | [mm] | [MPa] |
| 90:10 | M1% | 2.74 | 156.3 | 2.55 | 80.14 | 10.13 |
| 99:1 | M1% | 2.1 | 127.04 | 1.97 | 66.19 | 5.95 |

The creation of circular products through 3D printing was accomplished using recycled DCA-PU-M10% vitrimer-based filaments. The ability to recycle and reprocess these materials into various forms underscores their sustainability and versatility. To achieve suitable 3D-printable filaments, DCA-PU-M10% vitrimer foams were compounded with commercially available PLA or ABS pellets using a micro-compounder. Pure DCA-PU-M10% filaments exhibited insufficient printability due to inadequate mechanical consistency and elastomeric properties. However, blending with PLA or ABS significantly improved filament uniformity (e.g., at a 50:50 w/w ratio) and mechanical robustness, enabling successful extrusion and printability. These materials were combined using microcompounding twin-screw extrusion of blended PLA/ABS and DCA-PU feedstocks into 3D-printable filaments.

The observed compatibility between DCA-PU vitrimer foams and PLA/ABS suggests broader applicability in blending with other polymers to impart vitrimer properties. Preliminary 3D printing results confirm the successful fabrication of composite filaments with full 3D printability. These filaments were used to print dogbone samples for mechanical testing as well as a demonstration forming a UMass Lowell Riverhawk logo in FIG. 7.

This disclosure further encompasses the following aspects.

Aspect 1: A diol-containing monomer of the structure

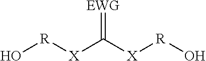

wherein X is independently at each occurrence sulfur (—S—) or nitrogen (—NH—); R is independently at each occurrence a C₁₋₁₂ alkylene group, a C₆₋₂₀ arylene group, a C₆₋₂₀ alkylarylene group, or a group of the formula —(CH₂CH₂O)_{y}CH₂CH₂—, wherein y is 1 to 4; and EWG is an electron withdrawing group.

Aspect 2: The diol-containing monomer of aspect 1, wherein EWG is selected from

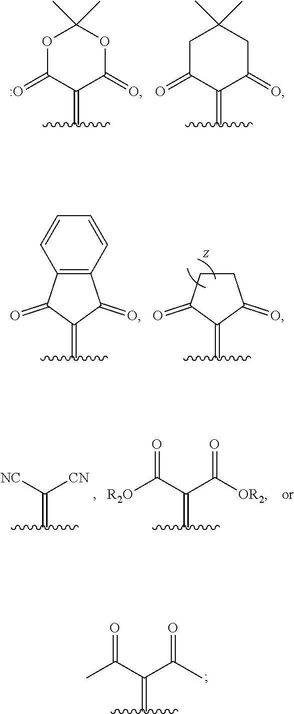

wherein z is 1 to 3; and R₂ is independently at each occurrence methyl or ethyl; wherein the curved lines represent points of attachment to the diol-containing monomer.

Aspect 3: The diol-containing monomer of aspect 1 or 2, wherein R is independently at each occurrence selected from

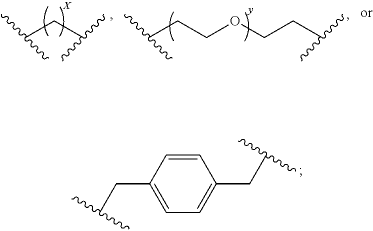

wherein x is 4 to 12; and y is 1 to 4; wherein the curved lines represent points of attachment to the diol-containing monomer.

Aspect 4: The diol-containing monomer of any of aspects 1 to 3, wherein the diol-containing monomer is of the structure

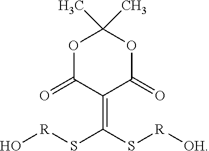

Aspect 5: The diol-containing monomer of any of aspects 1 to 3, wherein the diol-containing monomer is of the structure

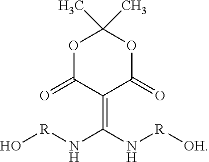

Aspect 6: The diol-containing monomer of any of aspects 1 to 3, wherein the diol-containing monomer is of the structure

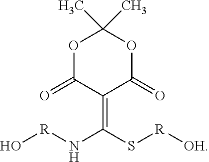

Aspect 7: The diol-containing monomer of any of aspect 2 1 to 3, wherein the diol-containing monomer is of the structure

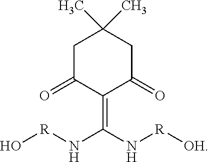

Aspect 8: The diol-containing monomer of any of aspects 1 to 3, wherein the diol-containing monomer is of the structure

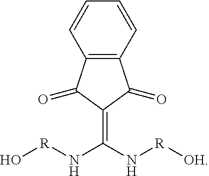

Aspect 9: The diol-containing monomer of any of aspects 1 to 8, wherein R is a C₁₋₁₂ alkylene group.

Aspect 10: The diol-containing monomer of any of aspects 1 to 9, wherein R is a C₁₋₆ alkylene group.

Aspect 11: The diol-containing monomer of any of aspects 1 to 10, wherein R is a C₃₋₆ alkylene group.

Aspect 12: A polyurethane comprising repeating units derived from the diol-containing monomer of any of aspects 1 to 11.

Aspect 13: The polyurethane of aspect 12, comprising repeating units derived from the diol-containing monomer of any of aspects 1 to 11; a diisocyanate; and optionally, a second diol-containing monomer.

Aspect 14: The polyurethane of aspect 13, wherein the diisocyanate comprises toluene diisocyanate, methylene diphenyl diisocyanate, or a combination thereof.

Aspect 15: The polyurethane of aspect 13 or 14, wherein the second diol-containing monomer comprises poly(tetramethylene glycol), hydroxy-terminated polybutadiene, or a combination thereof.

Aspect 16: The polyurethane of any of aspects 12 to 15, wherein the polyurethane is a crosslinked polyurethane.

Aspect 17: The polyurethane of aspect 16, wherein the crosslinked polyurethane comprises crosslinks derived from a triol, a tetraol, a trithiol, a tetrathiol, a triamine, a tetraamine, or a combination thereof.

Aspect 18: The polyurethane of aspect 17, wherein the crosslinks are derived from pentaerythritol, trimethylol propane, triethanol amine, tris(3-aminopropyl amine), or a combination thereof.

Aspect 19: The polyurethane of any of aspects 12 to 18, wherein the polyurethane is foamed.

Aspect 20: A reprocessable, crosslinked polyurethane comprising repeating units derived from the diol-containing monomer of any of aspects 1 to 11.

Aspect 21: An article comprising the reprocessable, crosslinked polyurethane of aspect 20.

Aspect 22: The article of aspect 21, wherein the article is a footwear component, preferably a shoe sole.

Aspect 23: A method for recycling a polyurethane, the method comprising: contacting a polyurethane comprising repeating units derived from the diol-containing monomer of any of aspects 1 to 11 with a decoupling agent under conditions effective to provide a polyurethane degradation product.

Aspect 24: The method of aspect 23, wherein the decoupling agent comprises at least one of a thiol group, a hydroxyl group, or an amine group.

Aspect 25: The method of claim 23 or 24, further comprising functionalizing the polyurethane degradation product.

Aspect 26: The method of any of claims 23 to 25, further comprising polymerizing the polyurethane degradation product to provide an upcycled polyurethane.

Aspect 27: An article comprising the upcycled polyurethane made according to the method of aspect 26, preferably wherein the article is a footwear component such as a shoe sole.

Aspect 28: A polyurethane vitrimer composition made by a method comprising: melt-processing a mixture comprising a first polyurethane comprising repeating units derived from the diol-containing monomer of any of aspects 1 to 11, and a second polyurethane; under conditions effective to provide the polyurethane vitrimer composition.

Aspect 29: The polyurethane vitrimer of aspect 28, wherein the mixture comprises 1 to 99 weight percent of the first polyurethane; and 1 to 99 weight percent of the second polyurethane, wherein weight percent is based on the total weight of the mixture.

Aspect 30: The polyurethane vitrimer of aspect 28 or 29, wherein the melt-processing comprises extrusion, injection molding, compression molding, or a combination thereof.

Aspect 31: A method of 3D printing, the method comprising: providing a molten composition comprising: a polyurethane comprising repeating units derived from the diol-containing monomer of any of aspects 1 to 11; and a processing aid comprising a thermoplastic polymer; extruding the molten composition to provide a fiber; forming an article from the fiber using an additive manufacturing technique.

Aspect 32: The method of aspect 31, wherein the additive manufacturing technique is fused filament fabrication.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other. “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “an aspect” means that a particular element described in connection with the aspect is included in at least one aspect described herein, and may or may not be present in other aspects. The term “combination thereof” as used herein includes one or more of the listed elements, and is open, allowing the presence of one or more like elements not named. In addition, it is to be understood that the described elements may be combined in any suitable manner in the various aspects.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“−”) that is not between two letters or symbols is used to indicate a point of attachment for a substituent. For example, —CHO is attached through carbon of the carbonyl group.

As used herein, the term “hydrocarbyl”, whether used by itself, or as a prefix, suffix, or fragment of another term, refers to a residue that contains only carbon and hydrogen. The residue can be aliphatic or aromatic, straight-chain, cyclic, bicyclic, branched, saturated, or unsaturated. It can also contain combinations of aliphatic, aromatic, straight chain, cyclic, bicyclic, branched, saturated, and unsaturated hydrocarbon moieties. However, when the hydrocarbyl residue is described as substituted, it may, optionally, contain heteroatoms over and above the carbon and hydrogen members of the substituent residue. Thus, when specifically described as substituted, the hydrocarbyl residue can also contain one or more carbonyl groups, amino groups, hydroxyl groups, or the like, or it can contain heteroatoms within the backbone of the hydrocarbyl residue. The term “alkyl” means a branched or straight chain, saturated aliphatic hydrocarbon group, e.g., methyl, ethyl, n-propyl, i-propyl, n-butyl, s-butyl, t-butyl, n-pentyl, s-pentyl, and n- and s-hexyl. “Alkenyl” means a straight or branched chain, monovalent hydrocarbon group having at least one carbon-carbon double bond (e.g., ethenyl (—HC═CH₂)). “Alkoxy” means an alkyl group that is linked via an oxygen (i.e., alkyl-O—), for example methoxy, ethoxy, and sec-butyloxy groups. “Alkylene” means a straight or branched chain, saturated, divalent aliphatic hydrocarbon group (e.g., methylene (—CH₂—) or, propylene (—(CH₂)₃—)). “Cycloalkylene” means a divalent cyclic alkylene group, —CₙH₂ₙ₋ₓ, wherein x is the number of hydrogens replaced by cyclization(s). “Cycloalkenyl” means a monovalent group having one or more rings and one or more carbon-carbon double bonds in the ring, wherein all ring members are carbon (e.g., cyclopentyl and cyclohexyl). “Aryl” means an aromatic hydrocarbon group containing the specified number of carbon atoms, such as phenyl, tropone, indanyl, or naphthyl. “Arylene” means a divalent aryl group. “Alkylarylene” means an arylene group substituted with an alkyl group. “Arylalkylene” means an alkylene group substituted with an aryl group (e.g., benzyl). The prefix “halo” means a group or compound including one more of a fluoro, chloro, bromo, or iodo substituent. A combination of different halo atoms (e.g., bromo and fluoro), or only chloro atoms can be present. The prefix “hetero” means that the compound or group includes at least one ring member that is a heteroatom (e.g., 1, 2, or 3 heteroatom(s)), wherein the heteroatom(s) is each independently N, O, S, Si, or P. “Substituted” means that the compound or group is substituted with at least one (e.g., 1, 2, 3, or 4) substituents that can each independently be a C₁₋₉ alkoxy, a C₁₋₉ haloalkoxy, a nitro (—NO₂), a cyano (—CN), a C₁₋₆ alkyl sulfonyl (—S(═O)₂-alkyl), a C₆₋₁₂ aryl sulfonyl (—S(═O)₂-aryl), a thiol (—SH), a thiocyano (—SCN), a tosyl (CH₃C₆H₄SO₂—), a C₃₋₁₂ cycloalkyl, a C₂₋₁₂ alkenyl, a C₅₋₁₂ cycloalkenyl, a C₆₋₁₂ aryl, a C₇₋₁₃ arylalkylene, a C₄₋₁₂ heterocycloalkyl, and a C₃₋₁₂ heteroaryl instead of hydrogen, provided that the substituted atom's normal valence is not exceeded. The number of carbon atoms indicated in a group is exclusive of any substituents. For example —CH₂CH₂CN is a C₂ alkyl group substituted with a nitrile.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A diol-containing monomer of the structure 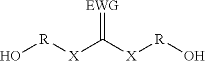
wherein
X is independently at each occurrence sulfur (—S—) or nitrogen (—NH—);
R is independently at each occurrence a C₁₋₁₂ alkylene group, a C₆₋₂₀ arylene group, a C₆₋₂₀ alkylarylene group, or a group of the formula —(CH₂CH₂O)_{y}CH₂CH₂—, wherein y is 1 to 4; and
EWG is an electron withdrawing group.

2. The diol-containing monomer of claim 1, wherein EWG is selected from 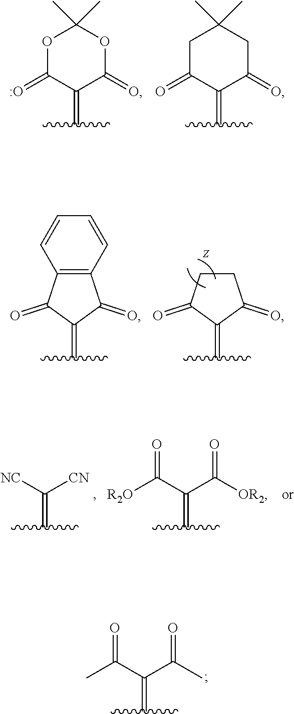
wherein
z is 1 to 3; and
R₂ is independently at each occurrence methyl or ethyl;
wherein the curved lines represent points of attachment to the diol-containing monomer.

3. The diol-containing monomer of claim 1, wherein R is independently at each occurrence selected from 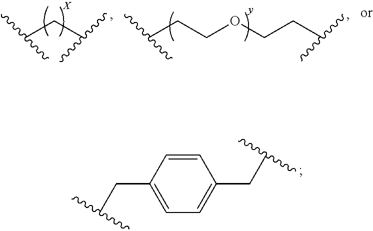
wherein
x is 4 to 12; and
y is 1 to 4;
wherein the curved lines represent points of attachment to the diol-containing monomer.

4. The diol-containing monomer of claim 1, wherein the diol-containing monomer is of the structure

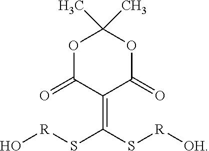

5. The diol-containing monomer of claim 1, wherein the diol-containing monomer is of the structure

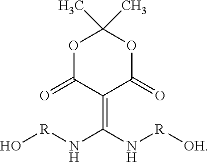

6. The diol-containing monomer of claim 1, wherein the diol-containing monomer is of the structure

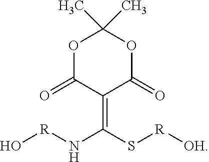

7. The diol-containing monomer of claim 1, wherein the diol-containing monomer is of the structure

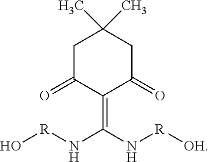

8. The diol-containing monomer of claim 1, wherein the diol-containing monomer is of the structure

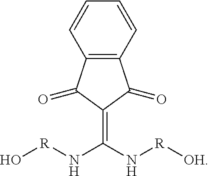

9. The diol-containing monomer of claim 1, wherein R is a C₁₋₁₂ alkylene group.

10. A polyurethane comprising repeating units derived from
the diol-containing monomer of claim 1;
a diisocyanate; and
optionally, a second diol-containing monomer.

11. The polyurethane of claim 10, wherein
the diisocyanate comprises toluene diisocyanate, methylene diphenyl diisocyanate, or a combination thereof, and
when present, the second diol-containing monomer comprises poly(tetramethylene glycol), hydroxy-terminated polybutadiene, or a combination thereof.

12. The polyurethane of claim 10, wherein the polyurethane is a crosslinked polyurethane, and the crosslinked polyurethane comprises crosslinks derived from a triol, a tetraol, a trithiol, a tetrathiol, a triamine, a tetraamine, or a combination thereof.

13. The polyurethane of claim 10, wherein the polyurethane is foamed.

14. A reprocessable, crosslinked polyurethane comprising repeating units derived from the diol-containing monomer of claim 1.

15. An article comprising the reprocessable, crosslinked polyurethane of claim 14, wherein the article is a footwear component.

16. A method for recycling a polyurethane, the method comprising:
contacting a polyurethane comprising repeating units derived from the diol-containing monomer of claim 1 with a decoupling agent comprising at least one of a thiol group, a hydroxyl group, or an amine group under conditions effective to provide a polyurethane degradation product.

17. The method of claim 16, further comprising polymerizing the polyurethane degradation product to provide an upcycled polyurethane.

18. A polyurethane vitrimer composition made by a method comprising:
melt-processing a mixture comprising
a first polyurethane comprising repeating units derived from the diol-containing monomer of claim 1, and
a second polyurethane;
under conditions effective to provide the polyurethane vitrimer composition.

19. The polyurethane vitrimer of claim 18, wherein the mixture comprises
1 to 99 weight percent of the first polyurethane; and
1 to 99 weight percent of the second polyurethane,
wherein weight percent is based on the total weight of the mixture.

20. The polyurethane vitrimer of claim 18, wherein the melt-processing comprises extrusion, injection molding, compression molding, or a combination thereof.
